(19) United States
(12) Patent Application Publication
(10) Pub. No.: US 2005/0287095 A1
(43) Pub. Date: Dec. 29, 2005

(54) Vesicle dispersion and cosmetic containing the same

(75) Inventor: Yukako Fujiwara, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KOSE Corporation, Tokyo (JP)

(21) Appl. No.: 10/518,549

(22) PCT Filed: Jul. 4, 2003
(86) PCT No.: PCT/JP03/08517

§ 371 Date: Jan. 3, 2005

(30) Foreign Application Priority Data
Jul. 5, 2002 (JP) 2002-196852

(51) Int. Cl. A61K 7/06; A61K 7/11

(52) U.S. Cl. 424/70.13

(57) ABSTRACT

A vesicle dispersion comprising (A) sucrose fatty acid ester, (B) a sphingosine and/or its derivative, and (C) an aqueous component and a cosmetic composition comprising the vesicle dispersion are disclosed. The vesicle dispersion can stably contain sphingosines such as a ceramide, excelling in a moisturizing effect of the skin, and the cosmetic composition comprising the vesicle dispersion exhibits an excellent moisturizing effect, storage stability, and the like.

### TECHNICAL FIELD

The present invention relates to a vesicle dispersion stably containing a component excelling in a moisturizing effect, a method for manufacturing the same, and cosmetic composition comprising the vesicle dispersion.

### BACKGROUND ART

Since intercellular lipids such as ceramide were confirmed to be deeply concerned with the barrier function of horny layers, development of moisturizing preparations comprising ceramide and the like has been pursued. However, because ceramide is highly crystalline, there has been a limitation to the amount of ceramide that can be incorporated into cosmetic compositions from the viewpoint of stability. Therefore, for the ceramide to more effectively exhibit the moisture retention function, development of a cosmetic composition which is stable even if a large amount of ceramide is incorporated and which does not produce problems such as crystal deposition has been desired.

To achieve the above object, a method of finely and stably incorporating lipids using a nonionic surfactant and an ionic surfactant in combination (Japanese Patent Application Laid-open-No. 4-193814), a method of making a liquid crystal of a lipid, a surfactant, and an oily agent (Japanese Patent Application Laid-open No. 6-345633), a method of depositing a lipid and a surfactant from an organic solvent and using the resulting complex (Japanese Patent Application Laid-open-No. 11-199462), a method of using liposome (vesicle of phospholipid bilayer), and the like have been studied.

However, since sphingosines such as ceramide have generally poor solubility in oil, the above methods require a comparatively large amount of surfactants and solvents, giving rise to a problem of safety as a cosmetic composition. In the case of a cosmetic composition in which the presence of an organic solvent is not preferred, a technology for completely removing a large amount of an organic solvent used for preparing a composite material is required for the above method of obtaining the composite material consisting of a lipid and a surfactant. In addition, since phospholipids are generally unstable materials, it has been difficult to ensure long-term storage stability as a cosmetic composition.

Therefore, development of a cosmetic composition excelling in a moisturizing effect, storage stability, and the like by stabilizing a system containing sphingosines such as ceramide over a long period of time has been desired.

### DISCLOSURE OF THE INVENTION

In view of this situation, the present inventors have conducted extensive studies on the means for stably incorporating sphingosines such as a ceramide and the like or their derivatives and found that a composition comprising a sucrose fatty acid ester, a sphingosine, and an aqueous component can provide a very stable vesicle structure without using an organic solvent. Consequently, sphingosines or their derivatives were successfully incorporated into cosmetic compositions and the like, resulting in products exhibiting non-sticky and excellent moisturizing feeling thereby completing the present invention.

Specifically, the present invention provides a vesicle dispersion comprising the following components (A), (B), and (C):
(A) a sucrose fatty acid ester,
(B) a sphingosine and/or its derivative, and
(C) an aqueous component.

The present invention also provides a cosmetic composition comprising the vesicle dispersion.

### BEST MODE FOR CARRYING OUT THE INVENTION

The term vesicle dispersion in the present invention refers to microcapsule of a lipid multilayer dispersed in an aqueous medium.

The sucrose fatty acid ester (component (A)), a component forming the vesicle dispersion of the present invention, may be any type of sucrose fatty acid ester which is commonly used for cosmetic compositions. Sucrose has eight hydroxyl groups in one molecule. These hydroxyl groups bond with fatty acid molecules to form a sucrose fatty acid ester. Any sucrose fatty acid ester having any degree of esterification (the substitution number of the hydroxyl groups with fatty acid) can be used in the present invention. Of these, however, monoesters, diesters, and triesters are preferable, with particularly preferable sucrose fatty acid esters being monoesters. Although a mixture of sucrose fatty acid esters with different degrees of esterification may also be used, 50 wt % (hereinafter indicated simply as “%”) or more of the component (A) is preferably sucrose fatty acid monoesters.

To ensure long term stability, it is desirable that all or a part of the component (A) be hydrophilic sucrose fatty acid esters. Specifically, the HLB of the component (A) is preferably in the range of 7-18, and particularly preferably in the range of 12-16.

The fatty acids forming the esters are preferably linear or branched, saturated or unsaturated fatty acids having 8-24 carbon atoms, preferably 14-20 carbon atoms.

It is more preferable that at least a part of these fatty acids be unsaturated fatty acids such as oleic acid and linolenic acid, since these unsaturated fatty acids act as an antioxidant on the skin and exhibit the effect of preventing aging.

Accordingly, palmitic acid, stearic acid, isostearic acid, oleic acid, and linolenic acid can be given as specific preferable examples of fatty acids forming the component (A).

As specific preferable examples of the component (A), sucrose monostearate, sucrose monoisostearate, sucrose diisostearate, sucrose monopalmitate, sucrose dipalmitate, sucrose monooleate, sucrose monolinolate, sucrose dilinolate, and sucrose trilinolate can be given.

Any compounds having a sphingosine skeleton can be used as the sphingosine and/or its derivative for forming the vesicle dispersion of the present invention (the component (B), hereinafter referred to as “sphingosines”), for example, phytosphingosine, ceramide, sphingomyelin, cerebroside, and the like can be given. These compounds may be used alone or in combination of two or more.

Among the compounds of the component (B), a ceramide generally has a high melting point and can be stably incorporated into a cosmetic composition only with difficulty. However, this difficulty can be overcome by the present invention and the use of ceramide as the component (B) is preferable to increase the moisture-retaining capability of the skin and the moisturizing effect of the cosmetic preparation.

Ceramides commonly used in cosmetics include those produced using yeast, chemically synthesized ceramides, and vegetable-origin ceramides, all of which can be preferably used in the present invention. Specific examples are ceramide 1 to ceramide 6. Of these, ceramide 2, ceramide 3, and ceramide 6 are particularly preferable.

As the aqueous component (component (C)) of the vesicle dispersion of the present invention, water or any compound soluble in water can be used. Examples include water, monohydric alcohols such as ethyl alcohol and isopropyl alcohol; glycols such as propylene glycol, 1,3-butylene glycol, dipropylene glycol, and polyethylene glycol; glycerols such as glycerin, diglycerol, and polyglycerol; and extracts from plants such as aloe vera, hamamelis, cucumber, lemon, lavender, and rose. Although these aqueous components may be used either alone or in combination of two or more, water or a mixture with water are preferable.

In addition to the above essential components, a fatty acid having a melting point of 80° C. or less and/or a higher alcohol having a melting point of 80° C. or less may be added as an optional component (component (D)) to the vesicle dispersion of the present invention. The component (D) can increase the mutual solubility of sphingosines and suppress crystal deposition, thereby promoting stability of the vesicle dispersion. Although either saturated or unsaturated, linear or branched fatty acids and higher alcohols having a melting point of 80° C. or less can be used as the component (D), branched fatty acids and higher alcohols are more preferable. Either one type or a mixture of two or more types of fatty acids and higher alcohols may be used. Specific examples include fatty acids such as isostearic acid and higher alcohols such as an isocetyl alcohol, isostearyl alcohol, and octyl dodecanol.

Furthermore, sterols may be added to the vesicle dispersion of the present invention as a component (E). The component (E) can promote stability of the vesicle dispersion and moisturizing effect of the skin. Any compound having a sterol skeleton or its derivative can be used as the component (E). As examples, cholesterols, phytosterols, macadamia nut oil fatty acid cholesteryl, coconut oil fatty acid cholesteryl, and N-lauroyl-L-glutamic acid di(cholesteryl behenyloctyldodecyl) can be given. Although these compounds may be used either individually or in combination of two or more, cholesterols and phytosterols are preferable.

In addition, at least one drug component selected from the group consisting of whitening agents, antiinflammatory agents, vitamins, amino acids, humectants, and antioxidants may be added to the vesicle dispersion of the present invention as a component (F).

The component (F) is an oil-soluble or water-soluble active component. Preferable examples are whitening agents such as ascorbic acid and its derivatives, and liquorice extract; antiinflammatory agents such as glycyrrhetinic acid and its derivatives, glycyrrhizic acid and its derivatives, and azulene; vitamins such as retinol, vitamin A derivatives, pyridoxine hydrochloride and its derivatives, nicotinic acid derivatives, vitamin E and its derivatives; amino acids such as histidine, arginine, and serine; humectants such as collagen, hyaluronic acid, and passive cutaneous anaphylaxis; and antioxidants such as butyl hydroxytoluene. One type or a mixture of two or more types can be used.

Preferred ranges of the above-descried components in the vesicle dispersion of the present invention are as follows.

| Component | Incorporated range | Preferred range |
|---|---|---|
| A | 0.1-20% | 2-10% |
| B | 0.01-5% | 0.1-2% |
| C | 62-99.9% | 83-97% |
| D | 0-5% | 0.1-2% |
| E | 0-3% | 0.01-1% |
| F | 0-5% | 0.01-2% |

The total amount of the component (A) and the component (B) is preferably 0.1-25% of the total amount of the vesicle dispersion.

Although not specifically limited, the ratio by weight of the component (B) to component (A) is preferably 0.001-0.4 from the view point of moisturizing effect and stability of the vesicle dispersion. A particularly preferable ratio is 0.01-0.2.

Although there are no specific limitations to the amount of the component (E), an amount in terms of the ratio by weight to the component (A), is preferably 0.001-0.4, and particularly preferably 0.1-0.2. The addition of the component (E) in an amount in this range can promote stability of the vesicle dispersion and moisturizing effect of the skin.

The vesicle dispersion of the present invention can be produced from the above components by various methods. The following method can be given as one example. Specifically, the components (A) and (B) and optionally the components (D) and (E) are dissolved or dispersed in the component (C) at 40° C. or higher. The resulting solution or dispersion is then added to the component (C), which may be either the same as or different from the component (C) used for the dissolution or dispersion, while stirring the mixture and controlling the temperature at 40° C. or higher.

A method of sufficiently swelling the components for forming a vesicle in water and stirring the mixture at a temperature of above Tc. (gel-liquid crystal transition temperature) (Japanese Patent No. 3126193), a method of forming a thin film of phospholipid using an organic solvent, adding water or an aqueous solution, and irradiating the thin film with ultrasonic radiation, for example, to obtain minute liposomes, and the like have been conventionally known as methods for manufacturing vesicle dispersions. The vesicle dispersion of the present invention, however, can be prepared easily without using an organic solvent by dissolving the vesicle components in a polyhydric alcohol such as dipropylene glycol or glycerin, for example, used as the component (C), and adding the solution to a component (C), which may not be the same component (C) used for dissolution, but contains water. A vesicle dispersion with a diameter of 0.2 mm or less can be easily obtained by using only a common stirrer.

Dipropylene glycol is particularly preferable as the component (C) used for preparing the above solution or dispersion among the above-mentioned compounds. As the component (C) to which the solution or dispersion is added, a mixture containing 20% or more of water is preferable, with a mixture containing water as a main component being particularly preferable. Use of the component (D) when preparing the above solution or dispersion can decrease the melting point of the component (B) and suppress deposition of crystals due to mutual solubility with the component (B), thereby promoting stability of the vesicle dispersion.

The vesicle dispersion of the present invention obtained in this manner can be combined with other cosmetic components to produce a cosmetic composition. The form of the cosmetic composition includes, but is not limited to, a solution-type, solubilizable-type, emulsion-type, oily-type, or aqueous-type, as well as a two layer type, three layer type, and the like consisting of two or three of these types. The cosmetic composition of the present invention can be a skin care cosmetic composition, a hair cosmetic composition, a make-up cosmetic composition, and the like, but is preferably a skin care cosmetic composition. Of the above forms, an aqueous-type preparation such as a lotion, emulsion, or cream is preferable due to the excellent moisturizing effect. The amount of the vesicle dispersion used in the cosmetic composition of the present invention is preferably 0.1-100%, although a specific amount varies depending on the type of cosmetic preparation.

In addition to the vesicle dispersion of the present invention, various optional components used for common cosmetic compositions, for example, water, a water soluble component, humectant, oil, surfactant, thickener, powder, coloring matter, UV absorber, film-forming agent, pH adjusting agent, discoloration inhibitor, antioxidant, antifoaming agent, beauty element, antiseptic agent, and perfume may be added to the cosmetic composition of the present invention, as appropriate.

As the water-soluble element, in addition to monoalcohols, glycols, glycerols, and extracts from plants, and the like previously mentioned as the component (C), saccharides such as sorbitol, maltitol, and sucrose and electrolytes such as sodium chloride, magnesium chloride, and sodium lactates can be used.

Proteins, mucopolysaccharides, collagen, elastin, and the like can be given as examples of the humectant.

As the oil, any oil, irrespective of origin (animal oils, vegetable oils, and synthetic oils) and properties (solid oils, half-solid oils, liquid oils, and volatile oils), such as hydrocarbons, oils and fats, waxes, hydrogenated oils, ester oils, fatty acids, higher alcohols, silicone oils, fluorine-containing oils, lanolin derivatives, and oily gelling agents can be used.

As the surfactant, any surfactant commonly used for cosmetics can be used.

As the thickener, water soluble polymers and the like, such as guar gum, sodium chondroitin sulfate, sodium hyaluronate, gum arabic, sodium alginate, carageenan, methylcellulose, hydroxyethylcellulose, carboxymethylcellulose, carboxyvinyl polymer, polyvinyl alcohol, polyvinyl pyrrolidone, and sodium polyacrylate can be given.

As the powder, inorganic fine particles, brightening fine particles, laminated film powder, organic fine particles, coloring-matter fine particles, composite fine particles, and the like can be used irrespective to their form (plate, spindle, acicular, or spherical), particle size, and particle structure (porous or non-porous), and the like. The surface of these fine particles may be treated with a fluorine compound, silicon containing oil agent, metallic soap, wax, surfactant, fat, oil, hydrocarbon, or the like by a conventional method.

As the UV absorber, a benzophenone-type, PABA-type, cinnamic acid-type, salicylic acid-type, 4-tert-butyl-4′-methoxy dibenzoylmethane, oxybenzone, and the like can be given as examples. As the film-forming agent, emulsion polymers such as alkyl (meth)acrylate copolymers can be given. As the pH adjusting agent, α-hydroxy acids such as lactic acid and citric acid, their salts, and edetates can be given. As the antioxidant, α-tocopherol, butylhydroxy toluene, and ascorbic acid can be given, for example. As examples of the beauty element, vitamins, antiphlogistics, and medicines such as herbal medicines can be mentioned. As the antiseptic agent, paraoxybenzoic acid, phenoxyethanol, and the like can be mentioned, for example.

The vesicle dispersion of the present invention obtained in this manner is a suspension of spherical vesicles in an aqueous medium, each vesicle having a concentric multilayer structure (onion-like structure) with an average particle diameter of 70-200 μm. It is possible to cause sphingosines such as a ceramide exhibiting an excellent moisturizing effect to the skin to be included in the vesicle in a stable manner.

Therefore, if the vesicle dispersion is incorporated into a cosmetic composition, not only the outstanding moisturizing effect possessed by sphingosines such as a ceramide can be exhibited, but also the storage stability can be increased.

### EXAMPLES

The present invention will be described in more detail with reference to Examples and Test Examples which should not be construed as limiting the present invention.

### Example 1

Vesicle Dispersion (1)

0.1 g of ceramide*¹ and 0.1 g of isostearic acid were weighed and the mixture was heated at 90° C. 4 g of dipropylene glycol in which 0.5 g of sucrose fatty acid ester*² was dispersed was added to this mixture and homogeneously mixed at 70° C. After the addition of 10 g of purified water at 70° C., the resulting mixture was stirred to disperse the components and cooled to obtain a vesicle dispersion, which contained the component (B) in the amount of 0.2 times the amount of component (A).
*1 Ceramide 2

*2 DK ester, S-160 (manufactured by Dai-ichi Kogyo Seiyaku Co., Ltd.)

### Example 2

Vesicle Dispersion (2)

0.01 g of ceramide*³ and 0.1 g of isostearic acid were weighed and the mixture was heated at 90° C. 4 g of dipropylene glycol in which 0.5 g of sucrose fatty acid ester*² was dispersed was added to this mixture and homogeneously mixed at 70° C. After the addition of 10 g of purified water at 70° C., the resulting mixture was stirred to disperse the components and cooled to obtain a vesicle dispersion, which contained the component (B) in the amount of 0.02 times the amount of component (A).
*2 The same as above.

*3 Ceramide 3

### Example 3

Vesicle Dispersion (3)

0.2 g of ceramide*¹ and 0.1 g of isostearic acid were weighed and the mixture was heated at 90° C. 4 g of dipropylene glycol in which 0.5 g of sucrose fatty acid ester*² was dispersed was added to this mixture and homogeneously mixed at 70° C. After the addition of 10 g of purified water at 70° C., the resulting mixture was stirred to disperse the components and cooled to obtain a vesicle dispersion, which contained the component (B) in the amount of 0.4 times the amount of component (A).
*1,*2 The same as above.

### Example 4

Vesicle Dispersion (4)

0.005 g of ceramide*¹, 0.005 g of phytosterol, and 0.1 g of isostearic acid were weighed and mixed, and the mixture was heated at 90° C. 4 g of dipropylene glycol in which 0.5 g of sucrose fatty acid ester*² was dispersed was added to this mixture and homogeneously mixed at 70° C. After the addition of 10 g of purified water at 70° C., the resulting mixture was stirred to disperse the components and cooled to obtain a vesicle dispersion, which contained the component (B) in the amount of 0.01 times the amount of component (A).
*1,*2 The same as above.

### Comparative Example 1

Vesicle Dispersion (5)

4 g of dipropylene glycol was added to a mixture of 0.1 g of isostearic acid and 0.5 g of sucrose fatty acid ester*² and the resulting mixture was homogeneously mixed at 70° C. The mixture was added to 5 g of purified water and the resulting mixture was stirred to disperse the components to obtain a vesicle dispersion.
*2 The same as above.

### Test Example 1

Vesicle Dispersion Evaluation Test

The dispersion stability and moisturizing effect of the vesicle dispersions obtained in Examples 1-4 and Comparative Example 1 were evaluated using the following methods. The results are shown in Table 1.

<Evaluation Methods>

a. Dispersion Stability

The samples were allowed to stand for one month in a thermostat at 40° C. to visually inspect deposition of crystals and the change of turbidity. The results were evaluated according to the following standard.

(Evaluation Standard)

\textcircled{O}: Neither crystal deposition nor turbidity were observed at all.
◯: Almost no crystal deposition and turbidity were observed.
Δ: Slight crystal deposition and turbidity were observed.
X: Crystal deposition and turbidity were clearly observed. (Precipitation or creaming was observed)
   b. Moisturizing Effect

Ten organoleptic panelists applied each sample to the upper arms to evaluate the moisturizing feeling after six hours. The results were classified into seven grades according to absolute criteria. The average of scores for each sample obtained by the absolute criteria was used for judgment according to the four-grade evaluation standard.

(1) Absolute Criteria

| (Score) | (Evaluation) |
|---|---|
| 6 | Excellent |
| 5 | Good |
| 4 | Slightly good |
| 3 | Fair |
| 2 | Slightly bad |
| 1 | Bad |
| 0 | Extremely bad |

(2) Four-Grade Evaluation Standard

| | | |
|---|---|---|
| Greater than 5: | Excellent: | ⊚ |
| Greater than 3 but less than or equal to 5: | Good: | ◯ |
| Greater than 2 but less than or equal to 3: | Slightly bad: | Δ |
| Less than or equal to 2: | Bad: | X |

TABLE 1

| | |
|---|---|
| Example | Comparative |

| | 1 | 2 | 3 | 4 | Example 1 |
|---|---|---|---|---|---|
| Vesicle dispersion | (1) | (2) | (3) | (4) | (5) |
| Evaluated item | | | | | |
| (a) Dispersion stability | ⊚ | ⊚ | ◯ | ⊚ | Δ |
| (b) Moisturizing effect | ⊚ | ◯ | ⊚ | ◯ | Δ |

The above results show that the vesicle dispersions (1)-(4) exhibited excellent dispersion stability and moisturizing effect. Consequently, the cosmetic compositions comprising these vesicle dispersions were evaluated to exhibit excellent dispersion stability and moisturizing effect.

### Example 5

Vesicle Face Lotion

Vesicle face lotions (Invention compositions 1-3) were prepared from the components shown in Table 2 using the following method of preparation.

TABLE 2

| | | Invention Composition No. | | |
|---|---|---|---|---|
| No. | Component | 1 | 2 | 3 |
| 1 | Vesicle dispersion (1) of | 15 | 5 | 30 |
| | Examples | | | |
| 2 | 1,3-butylene glycol | 6 | 6 | 6 |
| 3 | Glycerin | 5 | 5 | 5 |
| 4 | Citric acid | 0.1 | 0.1 | 0.1 |
| 5 | Sodium citrate | 0.2 | 0.2 | 0.2 |
| 6 | Purified water | balance* | balance* | balance* |
| 7 | POE (30) behenyl ether | 0.5 | 0.5 | 0.5 |
| 8 | Methyl p-oxybenzoate | q.s. | q.s. | q.s. |
| 9 | Ethyl alcohol | 8 | 8 | 8 |
| 10 | Perfume | q.s. | q.s. | q.s. |

*For the total amount of the composition.

(Method of Preparation)

The components 1-6 in Table 2 were mixed and dissolved. A solution obtained by mixing and dissolving the components 7-10 was added to the resulting solution and stirred to obtain vesicle face lotions.

### Comparative Example 2

Liposome Face Lotion

(1) 0.1 g of ceramide*¹, 0.05 g of isostearic acid, and 0.05 g of cholesterol were weighed and the mixture was heated at 90° C. 4 g of dipropylene glycol in which 0.5 g of phospholipid*⁴ was dispersed was added to this mixture and homogeneously mixed at 70° C. The mixture was added to 10 g of purified water at 70° C. After stirring to disperse the components, the resulting mixture was cooled to obtain a liposome solution.
*1 The same as above.

*4 Egg-yolk lecithin, PL-100P (manufactured by Q. P. Corp.)

(2) The liposome solution obtained in (1) above (15%), 1,3-butylene glycol (6%), glycerin (5%), citric acid (0.1%), sodium citrate (0.2%), and purified water (balance) were mixed and dissolved. A solution separately prepared from POE (30) behenyl ether (0.5%), ethanol (8%), and methyl p-oxybenzoate (q.s.), a perfume (q.s.), and purified water (balance) was added to the mixture, and stirred to obtain a liposome face lotion.

### Comparative Example 3

Emulsion Face Lotion

(1) 0.1 g of ceramide*¹ and 0.1 g of isostearic acid were weighed and mixed, and the mixture was heated at 90° C. 4 g of dipropylene glycol in which 0.5 g of polyoxyethylene (60E.O.) hydrogenated castor oil was dispersed was added to this mixture and homogeneously mixed at 70° C. The mixture was added to 10 g of purified water at 70° C. After stirring to disperse the components, the resulting mixture was cooled to obtain an emulsion.
*1 The same as above.

(2) The emulsion obtained in (1) above (15%), 1,3-butylene glycol (6%), glycerin (5%), citric acid (0.1%), sodium citrate (0.2%), and purified water (balance) were mixed and dissolved. A solution separately prepared from POE (30) behenyl ether (0.5%), ethanol (8%), and methyl p-oxybenzoate (q.s.), a perfume (q.s.), and purified water (balance) was added to the mixture, and stirred to obtain an emulsion face lotion.

### Test Example 2

The dispersion stability, moisturizing effect, non-stickiness, and change in odor of the vesicle face lotion (Invention product 1-3) of Example 5, the liposome face lotion of Comparative Example 2, and the emulsion face lotion of Comparative Example 3 were evaluated using the following methods. The results are shown in Table 3.

<Evaluation Methods>

a. Dispersion Stability

Measured in the same manner as in Test Example 1 and evaluated according to the same standard as in Test Example 1.

b. Moisturizing Effect and c. Non-Stickiness

Evaluated in the same manner and according to the same standard as the method of evaluating the moisturizing effect of Test Example 1, except that each face lotion was actually applied to the face.

d. Change in Odor

The face lotions were allowed to stand for one month in a thermostat at 40° C. After the temperature was reduced to the room temperature, the odor at the opening of the bottle was sensed to compare the odor of samples stored at room temperature. The results were evaluated according to the following standard.

(Evaluation standard)

\textcircled{O}: Almost no change in odor
◯: Some change in odor
Δ: Distinct odor change

X: Significant odor change

TABLE 3

| | | |
|---|---|---|
| Evaluated item | Invention product | Comparative |
| and results of | No. (Example 5) | Example |

| evaluation | 1 | 2 | 3 | 2 | 3 |
|---|---|---|---|---|---|
| a. Dispersion stability | ⊚ | ⊚ | ◯ | Δ | X |
| b. Moisturizing effect | ⊚ | ◯ | ⊚ | ⊚ | Δ |
| c. Non-stickiness | ⊚ | ⊚ | ◯ | Δ | ◯ |
| d. Change in odor | ◯ | ◯ | ◯ | X | ◯ |

All vesicle lotions of Example 5 exhibited an excellent moisturizing effect and excellent feeling of use as well as good dispersion stability. Their change in odor was within a tolerance level. Face lotions prepared in the same manner as in Example 5, wherein the vesicle dispersion (1) was replaced with the vesicle dispersions (2)-(4), were confirmed to be excellent in dispersion stability, moisturizing effect, non-stickiness, and odor change.

On the other hand, the liposome face lotion of Comparative Example 2 exhibited only poor non-stickiness and change in odor with passage of time, and the emulsion face lotion of Comparative Example 3 was inferior in dispersion stability and moisturizing effect.

### Example 6

Cream

Creams with the following composition were prepared according to the following method of preparation. Dispersion stability and moisturizing effect of the creams were evaluated.

| (Component) | (%) |
|---|---|
| 1. Stearic acid | 1.5 |
| 2. Cetostearyl alcohol | 3.0 |
| 3. Glyceryl monostearate | 1.5 |
| 4. Squalane | 20.0 |
| 5. Petroleum jelly | 5.0 |
| 6. Glycerin | 7.0 |
| 7. 1,3-Butylene glycol | 5.0 |
| 8. Vesicle dispersion of Examples*⁵ | 30.0 |
| 9. Sodium lactate | 1.0 |
| 10. Xanthan gum | 0.05 |
| 11. Antiseptic agent | q.s. |
| 12. Potassium hydroxide | 0.05 |
| 13. EDTA-2Na | 0.02 |
| 14. Purified water | balance* |
| 15. Perfume | q.s. |

*⁵Vesicle dispersions (1)-(4) were used.
*For the total amount of the composition.

(Method of Preparation)
A: Components 1-5 and 15 were mixed and heated at 70° C.
B: Components 6-14 were mixed and heated at 70° C.
C. B was added to A and the mixture was stirred and cooled to obtain a cream.

All creams of the Example 6 prepared using any one of vesicle dispersions (1)-(4) exhibited an excellent moisturizing effect and good stability.

### Example 7

Milky Lotion

Milky lotions with the following composition were prepared according to the following method of preparation. The dispersion stability and moisturizing effect of the milky lotions were evaluated.

| (Component) | (%) |
|---|---|
| 1. Squalane | 3.0 |
| 2. Dimethyl polysiloxane (20 cs) | 1.0 |
| 3. Polyoxyethylene (60) hydrogenated castor oil | 1.5 |
| 4. Cetostearyl alcohol | 0.3 |
| 5. Vesicle dispersion of Examples*⁵ | 15.0 |
| 6. Dipropylene glycol | 7.0 |
| 7. Glycerin | 5.0 |
| 8. Acrylic acid-alkyl methacrylate copolymer*¹⁰ | 0.1 |
| 9. Antiseptic agent | q.s. |
| 10. Sodium hydroxide | 0.03 |
| 11. EDTA-2Na | 0.02 |
| 12. Perfume | q.s. |
| 13. Purified water | balance* |

*⁵The same as above.
*¹⁰Pemulene TR-2(manufactured by NOVEON)
*For the total amount of the composition.

(Method of Preparation)
A: Components 1-7 were mixed and heated at 70° C.

B: Components 8-13 were mixed and heated at 70° C.

C. A was added to B and the mixture was stirred and cooled to obtain a milky lotion.

All milky lotions of the Example 7 prepared using any one of vesicle dispersion (1)-(4) exhibited an excellent moisturizing effect and good stability.

### Example 8

Eye Cream Stick

Eye cream sticks with the following composition were prepared according to the following method of preparation. The dispersion stability and moisturizing effect of the eye cream sticks were evaluated.

| (Component) | (%) |
|---|---|
| 1. Candelilla wax | 3.0 |
| 2. Polyethylene wax | 6.0 |
| 3. Micro crystalline wax | 2.5 |
| 4. Ceresin wax | 6.0 |
| 5. Paraffin | 10.0 |
| 6. Glyceryl triisooctanoate | 10.0 |
| 7. Liquid paraffin | balance* |
| 8. Silica fume | 1.0 |
| 9. Glycerin | 3.0 |
| 10. Vesicle dispersion of Examples*⁵ | 5.0 |

*⁵The same as above.
*For the total amount of the composition.

(Method of Preparation)
A: Components 1-7 were homogeneously dissolved at 100° C.
B. The components 8-10 were added to A and homogeneously dispersed.
C. B was poured into a stick container and solidified by cooling to obtain an eye cream stick.

All eye cream sticks of the Example 8 prepared using any one of vesicle dispersion (1)-(4) exhibited an excellent moisturizing effect and good stability.

### Example 9

Lipstick

Lipsticks with the following composition were prepared according to the following method of preparation and evaluated.

| (Component) | (%) |
|---|---|
| 1. Ethylene propylene copolymer | 5.0 |
| 2. Polyethylene wax | 5.0 |
| 3. Candelilla wax | 7.0 |
| 4. Acetic acid liquid lanolin | 10.0 |
| 5. Diglyceryl triisostearate | balance* |
| 6. Diglyceryl diisostearate | 3.0 |
| 7. Polybutene (molecular weight: 700) | 10.0 |
| 8. Liquid paraffin | 5.0 |
| 9. Vesicle dispersion of Examples*⁶ | 0.5 |
| 10. Vesicle dispersion of Examples*⁷ | 0.3 |
| 11. Red No. 202 | 0.1 |
| 12. Yellow No. 4 aluminum lake | 1.5 |
| 13. Titanium oxide | 2.0 |
| 14. Black iron oxide | 0.2 |
| 15. Silica fume | 3.0 |
| 16. Vitamin E | 0.5 |
| 17. Perfume | q.s. |

*⁶Vesicle dispersions (1)-(4) prepared in the same manner as in the method of preparing the vesicle dispersion (1), except that the amount of purified water was reduced to 5 g.
*⁷Vesicle dispersion obtained by the following method.
*For the total amount of the composition.

0.1 g of cerebroside and 0.1 g of cetyl alcohol were weighed and the mixture was heated at 70° C. 0.4 g of sucrose isostearic acid ester (hydrophilic), 0.1 g of sucrose linoleic ester (lipophilic), and 4 g of glycerin were added to this mixture and homogeneously mixed at 70° C. The mixture was added to 5 g of purified water at 70° C. After stirring to disperse the components, the resulting mixture was cooled.

(Method of Preparation)

A: Components 1-8 were homogeneously dissolved with heating.
B: The components 9-16 were added to A and the mixture was homogeneously mixed.
C: B was melted, filled into a die, and cooled to obtain a lip stick.

The lipsticks of Example 9 exhibited excellent non-stickiness and moisturizing feeling, and particularly imparted an excellent smooth feeling to the skin.

### Example 10

Beauty Lotions

Beauty lotions with the following composition were prepared according to the following method of preparation. The dispersion stability and moisturizing effect of the beauty lotions were evaluated.

| (Component) | (%) |
|---|---|
| 1. Vesicle dispersion of Examples*⁸ | 40.0 |
| 2. Vesicle dispersion of Examples*⁹ | 30.0 |
| 3. Ethyl alcohol | 3.0 |
| 4. Glycerin | 3.0 |
| 5. Sodium hyaluronate | 10.0 |
| 6. Purified water | balance* |
| 7. Perfume | q.s. |

*⁸Vesicle dispersions prepared in the same manner as the method of preparing the vesicle dispersion (1)-(4), except that sphingomyelin was used instead of ceramide and the amount of dipropylene glycol was reduced to half the amount.
*⁹Vesicle dispersion obtained by the following method.
*For the total amount of the composition.

0.1 g of ceramide*¹ and 0.1 g of isostearic acid were weighed and the mixture was heated at 90° C. 4 g of dipropylene glycol in which 0.4 g of sucrose fatty acid ester*² was dissolved, 0.1 g of sucrose linolenic ester, and 0.05 g of vitamin E were added and the resulting mixture was homogeneously mixed at 70° C. The mixture was added to 10 g of purified water in which 0.1 g of histidine was dissolved at 70° C. After stirring to disperse the components, the resulting mixture was cooled.
*1,*2 The same as above.

(Method of Preparation)

A: Components 1-7 were homogeneously mixed.

Beauty lotions of Example 10 prepared by using any combination of the four vesicle dispersions*⁸ with the vesicle dispersion*⁹ exhibited an excellent moisturizing effect and good stability.

### INDUSTRIAL APPLICABILITY

According to the present invention, a vesicle dispersion stably comprising sphingosines such as a ceramide can be easily obtained. This vesicle dispersion can be incorporated into a cosmetic composition and the like to provide the cosmetic composition and the like with excellent dispersion stability, moisturizing effect, non-stickiness, odor-change proofing effect, and the like.

1. A vesicle dispersion comprising the following components (A), (B), and (C):
(A) sucrose fatty acid ester,
(B) a sphingosine and/or its derivative, and
(C) an aqueous component.

2. The vesicle dispersion according to claim 1, wherein all or a part of the component (A) is hydrophilic sucrose fatty acid ester.

3. The vesicle dispersion according to claim 1, wherein 50 wt % or more of the component (A) is sucrose fatty acid monoester.

4. The vesicle dispersion according to claim 1, wherein a part of the component (A) is unsaturated fatty acid ester of sucrose.

5. The vesicle dispersion according to claim 4, wherein a part of the component (A) is γ-linolenic acid ester of sucrose.

6. The vesicle dispersion according to claim 1, wherein the component (B) is ceramide.

7. The vesicle dispersion according to claim 6, wherein the component (B) is chiral ceramide.

8. The vesicle dispersion according to claim 1, wherein the ratio by weight of the component (B) to the component (A) is 0.001-0.4.

9. The vesicle dispersion according to claim 1, further comprising a fatty acid having a melting point of 80° C. or less and/or a higher alcohol having a melting point of 80° C. or less as a component (D).

10. The vesicle dispersion according to claim 1, further comprising sterols as a component (E) in an amount, in terms of the ratio by weight to the component (A), of 0.001-0.4.

11. The vesicle dispersion according to claim 1, further comprising at least one drug component selected from the group consisting of whitening agents, antiinflammation agents, vitamins, amino acids, humectants, and antioxidants as a component (F).

12. The vesicle dispersion according to any one of claims 1 to 11, comprising 0.1-20 wt % of the component (A), 0.01-5 wt % of the component (B), 62-99.9 wt % of the component (C); 0-5 wt % of the component (D), 0-3 wt % of the component (E), and 0-5 wt % of the component (F) for the total of the vesicle dispersion.

13. The vesicle dispersion according to any one of claims 1 to 11, wherein the vesicle has an onion-like structure.

14. The vesicle dispersion according to claim 13, wherein the vesicle has an average particle diameter of 70-200 μm.

15. A cosmetic composition comprising the vesicle dispersion according to any one of claims 1 to 11.

16. A method for preparing the vesicle dispersion according to any one of claims 1 to 11, comprising dissolving or dispersing at least the component (A) and the component (B) in the component (C) containing a polyhydric alcohol at a temperature of 40° C. or higher, adding the resulting solution or dispersion to the component (C), which further contains water, while stirring and controlling the temperature at 40° C. or higher.
